(19) United States
(12) Patent Application Publication
(10) Pub. No.: US 2002/0042380 A1
(43) Pub. Date: Apr. 11, 2002

(54) Topical application of ascorbic acid compounds for augmenting the synthesis of epidermal ceramides

(76) Inventors: Isabelle Castiel, Jouy En Josas (FR); Corinne Ferraris, Paris (FR)

Correspondence Address: Norman H. Stepno, Esquire, BURNS, DOANE, SWECKER & MATHIS, L.L.P., P.O. Box 1404, Alexandria, VA, 22313-1404, US

(21) Appl. No.: 09/828,884

(22) Filed: Apr. 10, 2001

(30) Foreign Application Priority Data
Apr. 10, 2000 (FR) 00/04574

(51) Int. Cl. A61K 31/7048; A61K 31/665; A61K 31/375

(52) U.S. Cl. 514/24; 514/474; 514/99

(57) ABSTRACT

Vitamin C derivatives more stable than ascorbic acid itself and which more effectively increase the synthesis of epidermal ceramides, as well as improve the barrier function, moisture content and/or suppleness/surface appearance of the skin and which otherwise combat/prevent intrinsic aging thereof and are useful for the treatment of dermatitis, have the structural formula (I):

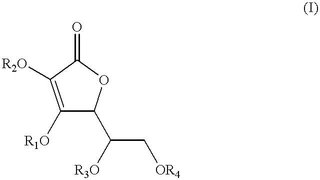

### CROSS-REFERENCE TO COMPANION APPLICATION

Copending application Ser. No. ______ [Attorney Docket No. 016800-440], filed concurrently herewith and assigned to the assignee hereof.

### CROSS-REFERENCE TO PRIORITY APPLICATION

This application claims priority under 35 U.S.C. §119 of FR-00/04574, filed Apr. 10, 2000, hereby expressly incorporated by reference.

### BACKGROUND OF THE INVENTION

1. Technical Field of the Invention

The present invention relates to the administration, via topical application onto human skin, of certain ascorbic acid (vitamin C) compounds/derivatives, or compositions comprised thereof, for, inter alia, increasing the synthesis of epidermal ceramides.

2. Description of the Prior Art

Human skin consists of two strata or layers, namely, a deep layer, the dermis, and a superficial layer, the epidermis.

The dermis provides the epidermis with a solid support. It is also its feeder component. It consists principally of fibroblasts and an extracellular matrix which is itself principally composed of collagen, elastin and a substance deemed the “ground” substance, these components being synthesized by the fibroblasts. Leukocytes, mastocytes or tissue macrophages are also present therein. The dermis also contains blood vessels and nerve fibers.

The epidermis is exposed to the external environment. Its role entails protecting the body from dehydration and from external factors, whether they are chemical, mechanical, physical or infectious attacks/challenges.

Natural human epidermis is composed mainly of three types of cells which are the keratinocytes, which are highly predominant, the melanocytes and the Langerhans' cells. Each of these cell types contributes, through its specific functions, to the essential role played by the skin in the organism.

The cells constituting the epidermis are delimited by a lipid domain. During differentiation, phospholipids, one role of which is to form the fluid structure of the cell membranes of the living layers of the epidermis, are progressively replaced by a mixture which is predominantly composed of fatty acids, cholesterol and sphingolipids.

These lipids are organized into specific lamellar structures whose integrity depends not only on the quality of the fractions present, but also on their respective proportions. This lamellar structure of the lipids of the lipid domain of the epidermis is responsible for the fluidity and, therefore, suppleness of the skin.

Lipids are also responsible for the “barrier” properties of the epidermis, particularly the stratum corneum.

Epidermal lipids are synthesized principally in the living epidermis. They essentially consist of phospholipids, sphingolipids, cholesterol, free fatty acids, triglycerides, esters of cholesterol and alkanes.

The phospholipids are essential for the constitution of the cell membranes. They play an important role in the mediation of extracellular signals and the formation of free aliphatic chains utilized for the production of energy. They constitute a reservoir of free fatty acids which are necessary for the constitution of sphingolipids.

Sphingolipids (or ceramides) are essential for maintaining the multilamellar structure of the intercorneocyte lipids. They are also essential for water-related exchanges and the “barrier” function of the epidermis.

Cholesterol plays a crucial role in skin hydration and in the “barrier” function of the epidermis.

And free fatty acids play a major role in maintaining the lamellar structure of the lipids of the stratum corneum, and in the constitution of the cell membranes where they are responsible for membrane fluidity and also for physiological processes such as receptor function or enzyme activity.

The essential role played by skin lipids and the importance which their integrity constitutes, are thus manifest.

However, in certain situations, whether in the event of specific pathological conditions (atopic dermatitis), skin aging, actinic aging, dry skin or else a barrier function which is impaired through repeated physical or chemical challenges, the human epidermis exhibits modifications in its lipid synthesis mechanism(s) and/or composition.

To improve the lipid content of the epidermis and consequently to favorably contribute to the suppleness of the skin, two mechanisms of action may be considered. The first is the exogeneous supply of lipid compounds by the topical route. The second entails stimulating the synthesis of endogeneous lipids. It has thus been demonstrated that it was possible to improve the lipid profile of reconstructed epidermes by adding ascorbic acid (vitamin C) to the culture medium (J. Invest. Dermatol., 109:348-355, 1997). However, because of its chemical structure (alpha-ketolactone), ascorbic acid is very sensitive to certain environmental parameters such as light, heat and aqueous media, in particular alkaline and/or aerobic media. Because of these problems of stability, it is necessary to use high concentrations of ascorbic acid in order to observe the effect, on the skin, of a composition comprised thereof.

Moreover, vitamin C derivatives are known which are more stable than ascorbic acid itself. Thus, EP-0,664,290 describes mono- and diesters of cinnamic acid or of one of its derivatives with ascorbic acid or derivative thereof. These compounds can be used as antioxidants in cosmetic or pharmaceutical compositions, in particular to protect the lipids in the skin against oxidation induced by free radicals. Other ascorbic acid derivatives, which are (5,6-isopropylidene)ascorbyl esters, are described in JP-46,024,699 and JP-44,000,220. Finally, other ascorbic acid compounds, such as the 2-O-α-D-glucopyranosyl of L-ascorbic acid (or ascorbyl glucoside), are commercially available. Ascorbyl glucoside is, in particular, a useful depigmenting agent.

### SUMMARY OF THE INVENTION

It has now unexpectedly and surprisingly been determined that a certain subgenus of ascorbic acid compounds, known to be more stable than ascorbic acid itself, exhibit greater efficacy than ascorbic acid in epidermal lipogenesis and thus can be administered for such purpose in smaller doses.

Briefly, then, the present invention features augmenting epidermal lipogenesis (increasing the synthesis of epidermal ceramides) by topically applying onto the skin of an individual subject in need of such treatment, an effective amount of at least one ascorbic acid compound having the structural formula (I):

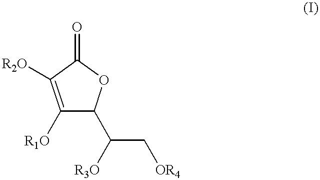

in which R₁ is a hydrogen atom, a sugar residue, a carbonyl group, an alkyloxycarbonyl radical or a carbamoyl radical, or, alternatively, R₁O is a sulfate functional group or a phosphate functional group; R₂ is a sugar residue or a radical —COR₅ wherein R₅ is (a) an optionally hydroxylated, linear, cyclic or branched, saturated or unsaturated C₁-C₂₀ hydrocarbon radical, (b) an optionally hydroxylated aryl radical, or (c) an aralkyl radical of formula (II):

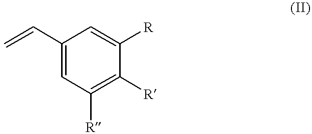

in which the radicals R, R′ and R″,which may be identical or different, are each a hydrogen atom, a hydroxyl, alkoxy, fluoroalkoxy or alkylcarbonyloxy radical; and R₃ and R₄, which may be identical or different, are each a hydrogen atom or a radical—COR₅ as defined above, with the proviso that R₃ and R₄ may together form an isopropylidene radical; or a salt of said at least one ascorbic acid compound (I).

### DETAILED DESCRIPTION OF BEST MODE AND SPECIFIC/PREFERRED EMBODIMENTS OF THE INVENTION

More particularly according to the present invention, in the compounds of formula (I) the radical R₁ is selected such that R₁O can be easily hydrolyzed upon contact with the skin.

By the expression “sugar residue” is preferably intended a glucose, galactose, mannose, fructose or N-acetylglucosamine residue.

By the expression “linear, cyclic or branched, saturated hydrocarbon radical having from 1 to 20 carbon atoms” is intended, for example, a radical selected from among methyl, ethyl, propyl, isopropyl, butyl, isobutyl, tert-butyl, hexyl, heptyl, 2-ethylhexyl, octyl, nonyl, isononyl, decyl, undecyl, dodecyl, pentadecyl, hexadecyl and octadecyl radicals, and preferably the undecyl radical.

And, in addition, by the expression “linear, cyclic or branched, unsaturated hydrocarbon radical having from 1 to 20 carbon atoms” is preferably intended a radical selected from among those radicals having from 2 to 5 carbon atoms and having one or more sites of ethylenic unsaturation, in particular the allyl radical.

The preferred substituted or unsubstituted aryl radical is the benzyl radical.

Exemplary aralkyl radicals having the above formula (II) are those in which the aryl nucleus is substituted with one or more hydroxyl radicals, or alkoxy radicals such as methoxy, ethoxy and butoxy radicals and preferably the methoxy radical, fluoroalkoxy radicals such as the trifluoromethoxy radical, and alkylcarbonyloxy radicals such as acetoxy, propionyloxy and butyryloxy radicals, and preferably acetoxy radicals.

The preferred ascorbic acid compounds according to the present invention are selected from the group consisting of:

(a) (5,6-isopropylidene)ascorbyl 2-hexadecanoate,

(b) (5,6-isopropylidene)ascorbyl 2-benzoate,

(c) 2-O-α-D-glucopyranosyl of (5,6- isopropylidene)ascorbic acid,

(d) ascorbyl 2-cinnamate,

(e) ascorbyl 2-ferulate,

(f) (5,6-isopropylidene)ascorbyl 2-ferulate, and

(g) (5,6-isopropylidene)ascorbyl 2-(4-acetoxyferulate).

(5,6-Isopropylidene)ascorbyl 2-hexadecanoate and (5,6-isopropylidene)ascorbyl 2-benzoate are particularly preferred.

The above compounds (a) and (b) and methodology for the preparation thereof are described in JP-46,024,699 and JP-44,000,220, respectively. The compound (c) is commercially available from HAYASHIBARA, under the trademark Ascorbic Acid 2-glucoside. The compounds (d), (e), (f) and (g) may be prepared as described in EP-0,664,290.

The aforementioned ascorbic acid compounds are well suited for formulation into compositions generally comprising a physiologically acceptable medium (vehicle, diluent or carrier). Such compositions advantageously contain from 0.001% to 10% by weight, and preferably from 0.01% to 1% by weight, of ascorbic acid compound(s).

Thus, it has now been demonstrated that the subject compounds are useful in a regime or regimen for increasing the synthesis of epidermal ceramides. By the expression “epidermal ceramides” according to the present description are intended both types I to VII ceramides, in particular types IV to VII ceramides, and acylglucosylceramides.

These properties of the ascorbic acid compounds/derivatives according to the invention on lipogenesis render the compositions comprised thereof particularly well suited for improving the barrier function of the skin, which permits better retention of water in the skin. The aforementioned ascorbic acid compounds are thus well suited for formulating therefrom moisturizing cosmetic compositions useful for moisturizing the skin.

More generally, the ascorbic acid derivatives according to the invention may be administered in a regime/regimen for cosmetic or dermatological purposes, as agents for improving the suppleness of the skin and/or the surface appearance of the skin (which manifests itself, in particular, by roughness and the presence of wrinkles and fine lines) and/or combating or preventing intrinsic aging of the skin. They are also useful for protecting the skin against a variety of challenges and attacks, in particular against the effects of chemical substances such as surfactants, irritant cosmetic agents such as retinoids, physical attacks such as rubbing, and also against the effects of the cold or the wind.

The cosmetic/dermatological compositions according to the invention are also useful for improving the barrier function of the skin. Such preparations may be for the treatment of certain pathological conditions involving disruption of the barrier function, such as atopic or seborrhoeic dermatitis.

In another embodiment of the invention, the aforesaid ascorbic acid compounds are administered to improve the content of lipids and/or the barrier function of reconstructed epidermes. The addition of at least one of these compounds to culture media for reconstructed epidermes thus makes it possible to ensure these epidermes resemble more closely the structure of normal human skin and thereby to permit the in vitro tests (in particular the penetration studies) carried out on these epidermes more predictive of the phenomena which will be observed in vivo.

The compositions of the invention may be formulated into any of the galenic forms normally employed for topical application, in particular in the form of an aqueous, aqueous/alcoholic or oily solution, an oil-in-water or water-in-oil or multiple emulsion, an aqueous or oily gel, an anhydrous liquid, pasty or solid product, a dispersion of oil in an aqueous phase with the aid of spherules, it being possible for these spherules to be polymeric nanoparticles such as nanospheres and nanocapsules, or more particularly lipid vesicles of the ionic and/or nonionic type.

The subject compositions may be fluid to a greater or lesser degree and may have the appearance of a white or colored cream, an ointment, a milk, a lotion, a serum, a paste, or a mousse or foam. They may optionally be applied onto the skin in the form of an aerosol. They may also be provided in the form of a solid, in particular in the form of lipstick when it is, for example, intended to treat chapped lips. Same can also be used for care products and/or makeup products for the skin.

In known fashion, the compositions of the invention may also contain the customary adjuvants and additives in the cosmetic field, such as hydrophilic or lipophilic gelling agents, hydrophilic or lipophilic bioaffecting active agents, preservatives, antioxidants, solvents, perfumes, fillers, UV-screening agents, pigments, odor absorbers and colorants. The amounts of these various adjuvants and additives are those conventionally used in the field considered, and range, for example, from 0.01% to 20% of the total weight of the composition. These additives and adjuvants, depending on their nature, may be introduced into the fatty phase, into the aqueous phase, into the lipid vesicles and/or into the nanoparticles. In any event, these adjuvants and additives, as well as their proportions, are selected such as not to impair the properties desired according to the invention.

When a composition of the invention is an emulsion, the proportion of the fatty phase may range from 5% to 80% by weight, and preferably from 5% to 50% by weight relative to the total weight of the composition. The oils, emulsifiers and coemulsifiers formulated into the emulsions are those conventionally employed in the field considered. The emulsifier and the coemulsifier are typically present in the subject compositions in a proportion ranging from 0.3% to 30% by weight, preferably from 0.5% to 20% by weight relative to the total weight of the composition.

Exemplary oils according to the invention include the mineral oils (liquid paraffin), oils of plant origin (avocado oil, soyabean oil), oils of animal origin (lanolin), synthetic oils (perhydrosqualene), silicone oils (cyclomethicone) and fluorinated oils (perfluoropolyethers). Exemplary fatty substances include the fatty alcohols (cetyl alcohol), fatty acids and waxes (carnauba wax, ozokerite).

Exemplary emulsifiers and coemulsifiers according to the invention include, for example, esters of a fatty acid and polyethylene glycol such as PEG-20 stearate, and esters of a fatty acid and glycerin such as glyceryl stearate.

Exemplary hydrophilic gelling agents include, in particular, carboxyvinyl polymers (carbomer), acrylic copolymers such as acrylate/alkyl acrylate copolymers, polyacrylamides, polysaccharides, natural gums and clays, and exemplary lipophilic gelling agents include the modified clays such as bentones, metal salts of fatty acids, hydrophobic silica and polyethylenes.

And exemplary active agents include, in particular, keratolytic and/or desquamatory agents, depigmenting agents, UV-screening agents, anti-free radical agents and mixtures thereof. In the event of incompatibility, at least some of the active agents may be incorporated into spherules, in particular ionic or nonionic vesicles or nanoparticles (nanocapsules and/or nanospheres), such that the active agents which are mutually incompatible are isolated from each other in the composition.

In order to further illustrate the present invention and the advantages thereof, the following specific examples are given, it being understood that same are intended only as illustrative and in nowise limitative.

### EXAMPLE 1

Effect of ascorbyl glucoside on the synthesis of epidermal lipids:

Ascorbyl glucoside and ascorbic acid itself were tested on a skin equivalent marketed by EPISKIN (Lyon, France), after culturing the latter for 7 days. The culture and assay media were those contained in the kit marketed by the supplier. Each compound was tested at 5 μg/ml and 0.05 μg/ml (as vitamin C equivalent) in the culture medium. The reconstructed epidermes were treated for five days, the culture medium being replaced every 48 hours. The control consisted of an identical epidermis equivalent which was subjected to topical application of culture medium without the compound to be tested.

At the end of the incubation period, the epidermis equivalent was detached from its collagenic support. The preparation of the lipids of the epidermis equivalent and their analysis by HPTLC or high-performance thin-layer chromatography were carried out according to the technique and with the buffers described by M. Ponec (1991, Adv. Lipid Res., 24:83-117). At the end of the migration, densitometric analysis was carried out using a CAMAG TLC model Scanner 3 densitometric reader.

Results and Conclusion

The results obtained are reported in the Table below as a percentage increase in ceramides 4, 5, 6 and 7 relative to the untreated control.

TABLE

| | | Skin treated |
|---|---|---|
| | Skin treated with | with ascorbyl |
| Concentration as vitamin C equiv. | ascorbic acid | glucoside |
| 5 μg/ml | | |
| Ceramides 4-7 | +21% | +40% |
| for which cer. 6-7 | −17% | +59% |
| 0.05 μg/ml | | |
| Cermides 4-7 | +7% | +11% |
| for which cer. 6-7 | +15% | +65% |

These results evidence an increase in the synthesis of the epidermal lipids (in particular of the most polar ceramides) which is much higher for ascorbyl glucoside than for ascorbic acid tested at the same concentration.

### EXAMPLE 2

The test described in Example 1 was repeated using various ascorbic acid derivatives, applied for 6 days at 1.10×10⁻⁴ M on reconstructed epidermes subjected to 7 days of culture. The quantity of epidermal ceramides produced was, however, not quantified. Instead, a densitometric analysis was carried out using a CAMAG TLC® model Scanner 3 densitometric reader.

The lipid profile of the treated epidermes was determined, in which:

Compound A was 2-O-α-D-glucopyranosyl of (5,6-isopropylidene)ascorbic acid;

Compound B was ascorbyl 2-cinnamate;

Compound C was ascorbyl 2-ferulate;

Compound D was (5,6-isopropylidene)ascorbyl 2-hexadecanoate;

Compound E was ascorbyl 2,3-bis(methoxycarbonyl-methoxy) ether;

Compound F was barium 2-ascorbyl sulfate; and

Compound G was (5,6-isopropylidene)ascorbyl 2-benzoate.

Compound E is described, as well as a process for the preparation thereof, in JP-07,206,840. Compound F is commercially available from SIGMA.

The lipid content was determined of the sample of reconstructed epidermis either untreated (control), or treated with ascorbic acid (vitamin C) or with one of the ascorbic acid derivatives A to G indicated above.

It will be appreciated from this study that the application, to the sample of reconstructed epidermis, of compounds A, B, C, D and G significantly increases the polar ceramide content of these epidermes, both relative to the control and relative to ascorbic acid. By contrast, compounds E and F, which are also ascorbic acid derivatives but which do not have the general formula (I) of the compounds according to the invention, elicit no significant effect on the epidermal lipogenesis.

The following are specific examples of compositions formulated according to this invention.

### EXAMPLE 3

Cosmetic Composition

The following composition was formulated conventionally:

| | |
|---|---|
| Octyldodecanol | 0.2% |
| Cyclomethicone | 5% |
| Dimethicone copolyol | 5% |
| Tocopheryl acetate | 1% |
| UV-screening agent | 1% |
| Ascorbyl glucoside | 0.1% |
| Glycerin | 3% |
| Disodium EDTA | 0.1% |
| pH-adjusting agents | 2.6% |
| Preservatives | 0.4% |
| Gelling agents | 1.2% |
| Water qs | 100% |

A fluid was obtained which may be applied in the morning and/or in the evening to the face to improve the suppleness of the skin and to smooth wrinkles and fine lines.

### EXAMPLE 4

Cosmetic Composition

An oil-in-water type emulsion was prepared by mixing the following ingredients:

| | |
|---|---|
| Ascorbyl 2-cinnamate | 0.1% |
| Oxyethylenated polyethylene glycol containing | 3% |
| 50 mol of EO | |
| Diglyceryl monostearate | 3% |
| Liquid paraffin | 24% |
| Cetyl alcohol | 5% |
| Water qs | 100% |

A cream was obtained which may be applied in the morning and/or in the evening to the face to improve the suppleness of the skin and to smooth wrinkles and fine lines.

### EXAMPLE 5

Cosmetic Composition

The following composition was formulated by mixing the following ingredients:

| | |
|---|---|
| Ascorbyl 2-ferulate | 0.1% |
| Jojoba oil | 13% |
| Mixture of methyl and propyl paraben | 0.05% |
| Potassium sorbate | 0.3% |
| Cyclopentadimethylsiloxane | 10% |
| Stearyl alcohol | 1% |
| Stearic acid | 4% |
| Polyethylene glycol stearate | 3% |
| Glycerol | 3% |
| Water qs | 100% |

A cream was obtained which is well suited for the treatment of dry skins.

In this example, the ascorbyl 2-ferulate may be replaced by the same amount of (5,6-isopropylidene)ascorbyl 2-ferulate or 5 (5,6-isopropylidene)ascorbyl 2-(4-acetoxyferulate).

### EXAMPLE 6

Cosmetic Composition

An oil-in-water type emulsion was prepared by mixing the following ingredients:

| | |
|---|---|
| (5,6-Isopropylidene)ascorbyl 2-hexadecanoate | 0.1% |
| Octyl palmitate | 10% |
| Glyceryl monoisostearate | 4% |
| Liquid paraffin | 24% |
| Vitamin E | 1% |
| Glycerol | 3% |
| Water qs | 100% |

A cream was obtained which may be applied in the morning and/or in the evening to the face to improve the suppleness of the skin and to smooth wrinkles and fine lines. In the above composition, (5,6-isopropylidene)ascorbyl 2-benzoate may be substituted for (5,6-isopropylidene)ascorbyl 2-hexadecanoate.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime/regimen for augmenting the synthesis of epidermal ceramides in the skin of an individual subject in need of such treatment, comprising topically applying onto said subject's skin, a thus-effective amount of at least one ascorbic acid compound having the structural formula (I): 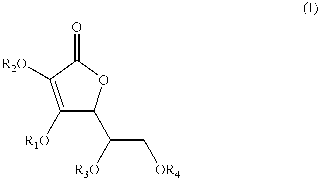
in which R₁ is a hydrogen atom, a sugar residue, a carbonyl group, an alkyloxycarbonyl radical or a carbamoyl radical, or, alternatively, R₁O is a sulfate functional group or a phosphate functional group; R₂ is a sugar residue or a radical —COR₅ wherein R₅ is (a) an optionally hydroxylated, linear, cyclic or branched, saturated or unsaturated C₁-C₂₀ hydrocarbon radical, (b) an optionally hydroxylated aryl radical, or (c) an aralkyl radical of formula (II): 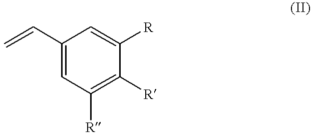
in which the radicals R, R′ and R″, which may be identical or different, are each a hydrogen atom, a hydroxyl, alkoxy, fluoroalkoxy or alkylcarbonyloxy radical; and R₃ and R₄, which may be identical or different, are each a hydrogen atom or a radical —COR₅ as defined above, with the proviso that R₃ and R₄ may together form an isopropylidene radical; or a salt of said at least one ascorbic acid compound (I).

2. The regime/regimen for augmenting the synthesis of epidermal ceramides as defined by claim 1, wherein formula (I) at least one of R₁ and/or R₂ is a glucose, galactose, mannose, fructose or N-acetylglucosamine residue.

3. The regime/regimen for augmenting the synthesis of epidermal ceramides as defined by claim 1, wherein formula (I) at least one of R₂, R₃ and/or R₄ is a radical —COR₅ in which R₅ is an optionally hydroxylated methyl, ethyl, propyl, isopropyl, butyl, isobutyl, tert-butyl, hexyl, heptyl, 2-ethylhexyl, octyl, nonyl, isononyl, decyl, undecyl, dodecyl, pentadecyl, hexadecyl or octadecyl radical.

4. The regime/regimen for augmenting the synthesis of epidermal ceramides as defined by claim 3, wherein formula (I) at least one of R₂, R₃ and/or R₄ is a radical —COR₅ in which R₅ is an optionally hydroxylated undecyl radical.

5. The regime/regimen for augmenting the synthesis of epidermal ceramides as defined by claim 1, wherein formula (I) at least one of R₂, R₃ and/or R₄ is a radical —COR₅ in which R₅ is an optionally hydroxylated linear or branched C₂-C₅ hydrocarbon having at least one site of ethylenic unsaturation.

6. The regime/regimen for augmenting the synthesis of epidermal ceramides as defined by claim 5, wherein formula (I) at least one of R₂, R₃ and/or R₄ is a radical —COR₅ in which R₅ is an optionally hydroxylated allyl radical.

7. The regime/regimen for augmenting the synthesis of epidermal ceramides as defined by claim 1, wherein formula (I) at least one of R₂, R₃ and/or R₄ is a radical —COR₅ in which R₅ is an aryl radical.

8. The regime/regimen for augmenting the synthesis of epidermal ceramides as defined by claim 7, wherein formula (I) at least one of R₂, R₃ and/or R₄ is a radical —COR₅ in which R₅ is a benzyl radical.

9. The regime/regimen for augmenting the synthesis of epidermal ceramides as defined by claim 1, wherein formula (I) at least one of R₂, R₃ and/or R₄ is a radical —COR₅ in which R₅ is an aralkyl radical of formula (II), in which at least one of R, R′ and/or R″ is an alkoxy radical.

10. The regime/regimen for augmenting the synthesis of epidermal ceramides as defined by claim 9, wherein formula (I) at least one of R₂, R₃ and/or R₄ is a radical —COR₅ in which R₅ is an aralkyl radical of formula (II), in which at least one of R, R′ and/or R″ is a methoxy, ethoxy or butoxy radical.

11. The regime/regimen for augmenting the synthesis of epidermal ceramides as defined by claim 10, wherein formula (I) at least one of R₂, R₃ and/or R₄ is a radical —COR₅ in which R₅ is an aralkyl radical of formula (II), in which at least one of R, R′ and/or R″ is a methoxy radical.

12. The regime/regimen for augmenting the synthesis of epidermal ceramides as defined by claim 1, wherein formula (I) at least one of R₂, R₃ and/or R₄ is a radical —COR₅ in which R₅ is an aralkyl radical of formula (II), in which at least one of R, R′ and/or R″ is a fluoroalkoxy radical.

13. The regime/regimen for augmenting the synthesis of epidermal ceramides as defined by claim 1, wherein formula (I) at least one of R₂, R₃ and/or R₄ is a radical —COR₅ in which R₅ is an aralkyl radical of formula (II), in which at least one of R, R′ and/or R″ is an alkylcarbonyloxy radical.

14. The regime/regimen for augmenting the synthesis of epidermal ceramides as defined by claim 13, wherein formula (I) at least one of R₂, R₃ and/or R₄ is a radical —COR₅ in which R₅ is an aralkyl radical of formula (II), in which at least one of R, R′ and/or R″ is an acetoxy, propionyloxy and butyryloxy radical.

15. The regime/regimen for augmenting the synthesis of epidermal ceramides as defined by claim 14, wherein formula (I) at least one of R₂, R₃ and/or R₄ is a radical —COR₅ in which R₅ is an aralkyl radical of formula (II), in which at least one of R, R′ and/or R″ is an acetoxy radical.

16. The regime/regimen for augmenting the synthesis of epidermal ceramides as defined by claim 1, said at least one ascorbic acid compound having the formula (I) comprising (5,6-isopropylidene)ascorbyl 2-hexadecanoate, (5,6-isopropylidene)ascorbyl 2-benzoate, the 2-O-α-D-glucopyranosyl of (5,6-isopropylidene)ascorbic acid, ascorbyl 2-cinnamate, ascorbyl 2-ferulate, (5,6-isopropylidene)ascorbyl 2-ferulate, or (5,6-isopropylidene)ascorbyl 2-(4-acetoxyferulate).

17. The regime/regimen for augmenting the synthesis of epidermal ceramides as defined by claim 16, said at least one ascorbic acid compound having the formula (I) being (5,6-isopropylidene)ascorbyl 2-hexadecanoate or (5,6-isopropylidene)ascorbyl 2-benzoate.

18. A regime/regimen for enhancing the barrier function of the skin of an individual subject in need of such treatment, comprising topically applying onto said subject's skin, a thus-effective amount of at least one ascorbic acid compound having the structural formula (I): 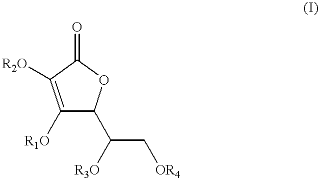
in which R₁ is a hydrogen atom, a sugar residue, a carbonyl group, an alkyloxycarbonyl radical or a carbamoyl radical, or, alternatively, R₁O is a sulfate functional group or a phosphate functional group; R₂ is a sugar residue or a radical —COR₅ wherein R₅ is (a) an optionally hydroxylated, linear, cyclic or branched, saturated or unsaturated C₁-C₂₀ hydrocarbon radical, (b) an optionally hydroxylated aryl radical, or (c) an aralkyl radical of formula (II): 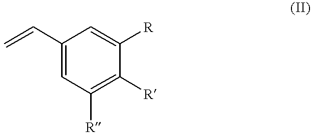
in which the radicals R, R′ and R″, which may be identical or different, are each a hydrogen atom, a hydroxyl, alkoxy, fluoroalkoxy or alkylcarbonyloxy radical; and R₃ and R₄, which may be identical or different, are each a hydrogen atom or a radical —COR₅ as defined above, with the proviso that R₃ and R₄ may together form an isopropylidene radical; or a salt of said at least one ascorbic acid compound (I).

19. A regime/regimen for the treatment of atopic or seborrhoeic dermatitis of the skin of an individual subject in need thereof, comprising topically applying onto said subject's skin, a thus-effective amount of at least one ascorbic acid compound having the structural formula (I): 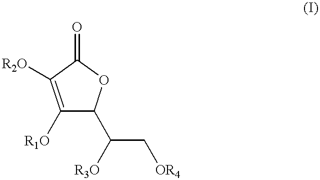
in which R₁ is a hydrogen atom, a sugar residue, a carbonyl group, an alkyloxycarbonyl radical or a carbamoyl radical, or, alternatively, R₁O is a sulfate functional group or a phosphate functional group; R₂ is a sugar residue or a radical —COR₅ wherein R₅ is (a) an optionally hydroxylated, linear, cyclic or branched, saturated or unsaturated C₁-C₂₀ hydrocarbon radical, (b) an optionally hydroxylated aryl radical, or (c) an aralkyl radical of formula (II): 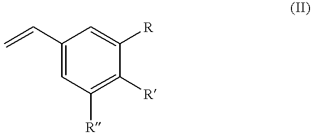
in which the radicals R, R′ and R″, which may be identical or different, are each a hydrogen atom, a hydroxyl, alkoxy, fluoroalkoxy or alkylcarbonyloxy radical; and R₃ and R₄, which may be identical or different, are each a hydrogen atom or a radical —COR₅ as defined above, with the proviso that R₃ and R₄ may together form an isopropylidene radical; or a salt of said at least one ascorbic acid compound (I).

20. A regime/regimen for improving the suppleness of the skin and/or the surface appearance of the skin and/or combating or preventing intrinsic aging of the skin of an individual subject in need of such treatment, comprising topically applying onto said subject's skin, a thus-effective amount of at least one ascorbic acid compound having the structural formula (I): 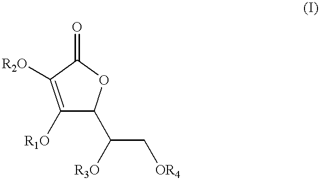
in which R₁ is a hydrogen atom, a sugar residue, a carbonyl group, an alkyloxycarbonyl radical or a carbamoyl radical, or, alternatively, R₁O is a sulfate functional group or a phosphate functional group; R₂ is a sugar residue or a radical —COR₅ wherein R₅ is (a) an optionally hydroxylated, linear, cyclic or branched, saturated or unsaturated C₁-C₂₀ hydrocarbon radical, (b) an optionally hydroxylated aryl radical, or (c) an aralkyl radical of formula (II): 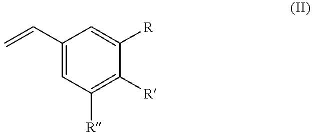
in which the radicals R, R′ and R″, which may be identical or different, are each a hydrogen atom, a hydroxyl, alkoxy, fluoroalkoxy or alkylcarbonyloxy radical; and R₃ and R₄, which may be identical or different, are each a hydrogen atom or a radical —COR₅ as defined above, with the proviso that R₃ and R₄ may together form an isopropylidene radical; or a salt of said at least one ascorbic acid compound (I).

21. A regime/regimen for moisturizing the skin of an individual subject in need of such treatment, comprising topically applying onto said subject's skin, a thus-effective amount of at least one ascorbic acid compound having the structural formula (I): 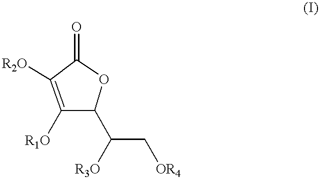
in which R₁ is a hydrogen atom, a sugar residue, a carbonyl group, an alkyloxycarbonyl radical or a carbamoyl radical, or, alternatively, R₁O is a sulfate functional group or a phosphate functional group; R₂ is a sugar residue or a radical —COR₅ wherein R₅ is (a) an optionally hydroxylated, linear, cyclic or branched, saturated or unsaturated C₁-C₂₀ hydrocarbon radical, (b) an optionally hydroxylated aryl radical, or (c) an aralkyl radical of formula (II): 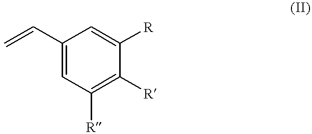
in which the radicals R, R′ and R″, which may be identical or different, are each a hydrogen atom, a hydroxyl, alkoxy, fluoroalkoxy or alkylcarbonyloxy radical; and R₃ and R₄, which may be identical or different, are each a hydrogen atom or a radical —COR₅ as defined above, with the proviso that R₃ and R₄ may together form an isopropylidene radical; or a salt of said at least one ascorbic acid compound (I).

22. A method for enhancing the lipid content and/or improving the barrier function of a reconstructed epidermis, comprising including therein a thus-effective amount of at least one ascorbic acid compound having the structural formula (I): 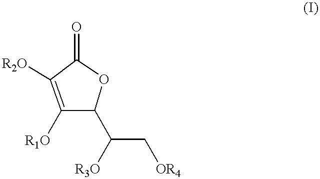
in which R₁ is a hydrogen atom, a sugar residue, a carbonyl group, an alkyloxycarbonyl radical or a carbamoyl radical, or, alternatively, R₁O is a sulfate functional group or a phosphate functional group; R₂ is a sugar residue or a radical —COR₅ wherein R₅ is (a) an optionally hydroxylated, linear, cyclic or branched, saturated or unsaturated C₁-C₂₀ hydrocarbon radical, (b) an optionally hydroxylated aryl radical, or (c) an aralkyl radical of formula (II): 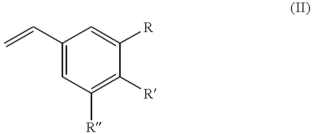
in which the radicals R, R′ and R″, which may be identical or different, are each a hydrogen atom, a hydroxyl, alkoxy, fluoroalkoxy or alkylcarbonyloxy radical; and R₃ and R₄, which may be identical or different, are each a hydrogen atom or a radical —COR₅ as defined above, with the proviso that R₃ and R₄ may together form an isopropylidene radical; or a salt of said at least one ascorbic acid compound (I).

23. A topically applicable cosmetic/dermatological composition suited for augmenting the synthesis of epidermal ceramides, and/or improving the barrier function of the skin, and/or treating atopic or seborrhoeic dermatitis, and/or improving the suppleness and/or surface appearance of the skin and/or combating or preventing intrinsic aging of the skin, and/or moisturizing the skin, comprising a thus-effective amount of at least one ascorbic acid compound having the structural formula (I): 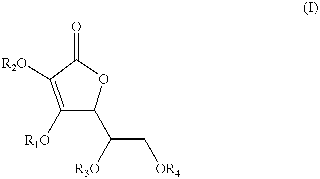
in which R₁ is a hydrogen atom, a sugar residue, a carbonyl group, an alkyloxycarbonyl radical or a carbamoyl radical, or, alternatively, R₁O is a sulfate functional group or a phosphate functional group; R₂ is a sugar residue or a radical —COR₅ wherein R₅ is (a) an optionally hydroxylated, linear, cyclic or branched, saturated or unsaturated C₁-C₂₀ hydrocarbon radical, (b) an optionally hydroxylated aryl radical, or (c) an aralkyl radical of formula (II): 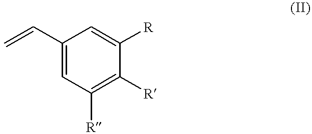
in which the radicals R, R′ and R″, which may be identical or different, are each a hydrogen atom, a hydroxyl, alkoxy, fluoroalkoxy or alkylcarbonyloxy radical; and R₃ and R₄, which may be identical or different, are each a hydrogen atom or a radical —COR₅ as defined above, with the proviso that R₃ and R₄ may together form an isopropylidene radical; or a salt of said at least one ascorbic acid compound (I); formulated into a topically applicable, physiologically acceptable vehicle, diluent or carrier therefor.

24. The topically applicable cosmetic/dermatological composition as defined by claim 23, comprising from 0.001% to 10% by weight of said at least one ascorbic acid compound, or salt thereof.

25. The topically applicable cosmetic/dermatological composition as defined by claim 24, comprising from 0.01% to 1% by weight of said at least one ascorbic acid compound, or salt thereof.

26. The topically applicable cosmetic/dermatological composition as defined by claim 23, comprising an aqueous, aqueous/alcoholic or oily solution, or dispersion.

27. The topically applicable cosmetic/dermatological composition as defined by claim 23, comprising an emulsion, a gel, a paste, a solid, spherules, lipid vesicles, a cream, a lotion, a milk, a mousse or foam, a serum, an aerosol, an ointment, or a makeup.

28. The topically applicable cosmetic/dermatological composition as defined by claim 23, further comprising at least one hydrophilic or lipophilic gelling agent, hydrophilic or lipophilic bioaffecting active agent, preservative, antioxidant, solvent, perfume, filler, UV-screening agent, pigment, odor absorber, colorant or combination thereof.
